(19) United States
(12) Patent Application Publication
(10) Pub. No.: US 2008/0187589 A1
(43) Pub. Date: Aug. 7, 2008

(54) MULTI-MODAL DELIVERY VIA TRANSMUCOSAL AND GASTRO-INTESTINAL ABSORPTION OF ANTIHISTAMINES AND SYMPTOM RELIEF

(76) Inventors: JOHN M. PINNEY, St. Michaels, MD (US); Edward J. Cone, Severna Park, MD (US); Tommy L. Chau, Ashburn, VA (US); August R. Buchhalter, Frederick, MD (US)

Correspondence Address: BERENATO, WHITE & STAVISH, LLC, 6550 ROCK SPRING DRIVE, SUITE 240, BETHESDA, MD, 20817, US

(21) Appl. No.: 12/026,303

(22) Filed: Feb. 5, 2008

Related U.S. Application Data
(60) Provisional application No. 60/899,400, filed on Feb. 5, 2007.

(51) Int. Cl. A61K 9/24; A61K 9/20; A61P 37/08; A61K 31/4545

(52) U.S. Cl. 424/472; 424/464; 514/290

(57) ABSTRACT

A multi-modal absorption, medicine-delivery lozenge is provided. The lozenge includes a lozenge base for oral administration into a user's mouth, a medicinal active ingredient including an antihistamine convertible between ionized and non-ionized states, and a buffer present in an effective amount for establishing a pH in the individual's mouth which is characterized by a simultaneous presence of a first pharmacologically effective dose of the non-ionized form of the medicinal active ingredient for transmucosal absorption, and a second pharmacologically effective dose of the ionized form of the medicinal active ingredient for gastro-intestinal absorption.

### CROSS-REFERENCE TO RELATED APPLICATION AND CLAIM OF PRIORITY

This application claims the benefit of priority under 35 U.S.C. § 119 of U.S. provisional application Ser. No. 60/899,400 filed Feb. 5, 2007, the complete disclosure of which is incorporated herein by reference.

### FIELD OF THE INVENTION

The present invention relates to a multi-modal absorption, medicine-delivery lozenge containing an orally administered medication that is released in the oral cavity for rapid transmucosal absorption and long-term gastro-intestinal absorption. The present invention further relates to a method of relieving histamine effects and symptoms and a method of relieving allergic responses.

### BACKGROUND OF THE INVENTION

Common allergic reactions and symptoms suffered by those with allergies include nasal congestion, sneezing, “running noses,” and headaches. Allergic reactions result from the release of histamine from mast cells lining nasal mucous membranes. The released histamine binds to local histamine receptors to constrict bronchial smooth muscle and dilate blood vessels.

Antihistamines relieve histamine effects by preemptively binding to the histamine receptors, thereby blocking histamines from binding to the receptors. Antihistamines are often administered in combination with sympathomimetic or decongestant drugs. Sympathomimetic drugs such as pseudoephedrine, phenylephrine and phenylpropanolamine are recognized as therapeutic agents for nasal congestion relief.

In most oral delivery systems active ingredient(s) are swallowed for absorption in the gastrointestinal (GI) system. Although oral delivery is non-invasive and convenient, most medications are not well-absorbed in the stomach and must transit into the gut for complete absorption. Many medications are subject to a “first-pass effect” during GI absorption. Active drug-metabolizing enzymes in the gut wall and in the liver can convert a large portion of medication(s) to inactive and sometimes, active metabolites. Unabsorbed drug(s) pass through the GI system and are excreted. The active portion of the drug that enters the bloodstream may represent only a small fraction of the amount of active ingredient originally present in the oral product. Consequently, frequently oral formulations contain more active ingredient than is needed to account for the losses that occur from metabolic inactivation during absorption and/or from excretion of unabsorbed active ingredient(s).

Further, there is substantial inter-individual variability in gastrointestinal absorption rates. Some individuals absorb only sub-therapeutic amounts of medicine in the gastrointestinal tract. To ensure that individuals having poor GI absorption absorb the amount of active ingredient needed, the administered serving may contain excess active ingredient. However, those individuals better able to absorb the active ingredient in the gastrointestinal tract may absorb excess active ingredient, resulting in the development of avoidable side-effects.

Also, the transit of medicine through the GI system requires a substantial amount of time. The degree of absorption of active ingredients can be dependent upon numerous factors, including dissolution times, gastric emptying time, amount of liquid present, and influence of food upon absorption. The time-delay for production of effective blood levels of active ingredients by the oral route generally ranges from 30 minutes to four hours. Therefore, relief of symptoms is substantially delayed.

Some active ingredients can be absorbed directly through the oral mucosa via buccal and sublingual routes, while other active ingredients display chemical properties that prevent transmucosal absorption. Generally, active ingredients can be classified by their chemical makeup as acids, neutrals or bases. Acids can be neutralized by basic chemicals to form corresponding salts. Bases can be neutralized by acidic chemicals to form corresponding salts. Generally, the salt forms of active ingredients display greater water solubility and less lipid solubility than the corresponding unreacted form. Either the free, unreacted form or the salt form of active ingredients may be placed in the delivery vehicle for release into the mouth. In some cases, the active ingredient may be trapped or bound in either the ionized or non-ionized form in a vehicle, e.g., resins. Upon release from the delivery vehicle into the mouth, some active ingredients will be highly ionized as a result of the pH characteristics of oral fluids (saliva) while others will exist primarily in the neutral or non-ionized form. Once the active ingredient is released into the oral cavity, only the non-ionized form of the active ingredient(s) will be absorbed through the oral mucosa. The fraction of non-ionized active ingredient(s) that is present in saliva will determine the total amount that can be absorbed.

Absorption of active ingredient(s) through the oral mucosa into the bloodstream effectively bypasses the degradation that occurs in the gut wall and liver and provides an alternate means of providing active ingredients. Consequently, higher bioavailability of active ingredients may be achieved by transmucosal delivery than by GI ingestion. An additional advantage of buccal/sublingual delivery of active ingredients is the rapid delivery of unmetabolized active ingredient(s) to the bloodstream. Under suitable pH conditions, active ingredients introduced into the oral cavity may be rapidly absorbed and can appear in the bloodstream within minutes of application.

Despite the various attempts in the art to incorporate active ingredients in a base material, none have been satisfactory, in part, because of inadequate early release of active ingredient or inadequate control of pH conditions. Tablets, liquids and oral sprays have not sufficiently controlled the pH conditions of the mouth. Consequently, much of the dose is ultimately swallowed and exposed to the degradative processes of the gastrointestinal system.

### SUMMARY OF THE INVENTION

A first aspect of the invention provides a multi-modal absorption, medicine-delivery lozenge, featuring a lozenge base for oral administration into a user's mouth, a medicinal active ingredient including an antihistamine convertible between ionized and non-ionized forms, and a buffer present in an effective amount for establishing a pH in the individual's mouth which is characterized by a simultaneous presence of a first pharmacologically effective dose of the non-ionized form of the medicinal active ingredient for transmucosal absorption, and a second pharmacologically effective dose of the ionized form of the medicinal active ingredient for gastro-intestinal absorption.

According to a second aspect of the invention, a method of attaining relief from histamine effects and symptoms is provided, in which a multi-modal absorption, medicine-delivery lozenge is administered to an individual in need of relief of histamine effects and symptoms. The lozenge contains a lozenge base for oral administration into the individual's mouth, a medicinal active ingredient including an antihistamine convertible between ionized and non-ionized forms, and a buffer present in an effective amount for establishing a pH in the individual's mouth which is characterized by a simultaneous presence of a first pharmacologically effective dose of the non-ionized form of the medicinal active ingredient for transmucosal absorption, and a second pharmacologically effective dose of the ionized form of the medicinal active ingredient for gastrointestinal absorption.

A third aspect of the invention provides a method of attaining relief from allergic responses. The method involves orally administering a multi-modal absorption, medicine-delivery lozenge to an individual in need of relief of allergic responses. The lozenge contains a lozenge base for oral administration into the individual's mouth, a medicinal active ingredient including an antihistamine convertible between ionized and non-ionized forms, and a buffer present in an effective amount for establishing a pH in the individual's mouth which is characterized by a simultaneous presence of a first pharmacologically effective dose of the non-ionized form of the medicinal active ingredient for transmucosal absorption, and a second pharmacologically effective dose of the ionized form of the medicinal active ingredient for gastro-intestinal absorption.

Other aspects of the invention will become more apparent from a reading of the following detailed description of exemplary embodiments and exemplary methods of the invention.

### BRIEF DESCRIPTION OF THE DRAWING

The accompanying drawing is incorporated in and constitutes a part of the specification. The drawing, together with the general description given above and the detailed description of the exemplary embodiment(s) and method(s) given below, serve to explain the principles of the invention. In such drawing:

FIG. 1 is a graph showing theoretical plasma concentrations following administration of 10 mg of loratadine administered by transmucosal/oral versus oral routes.

### DETAILED DESCRIPTION OF EXEMPLARY EMBODIMENT(S) AND EXEMPLARY METHOD(S) OF THE INVENTION

Reference will now be made in detail to exemplary embodiment(s) and method(s) of the invention as illustrated in the accompanying drawings. It should be noted, however, that the invention in its broader aspects is not limited to the specific details, representative devices and methods, and illustrative examples shown and described in this section in connection with the exemplary embodiments and methods.

A multi-modal absorption, medicine-delivery lozenge according to an embodiment of the invention contains a lozenge base, an active medicinal ingredient, and a buffer for controlling the pH of fluid (saliva) in the user's mouth to facilitate dissolution and transmucosal absorption of the active medicinal ingredient. In exemplary embodiments of the invention, the lozenge provides bi-modal absorption comprising a first pharmacologically effective dose of medicine for rapid transmucosal absorption, and a second pharmacologically sustained dose for longer-term absorption to relieve symptoms and allergic effects, and/or for providing therapeutic treatment.

Lozenge Base (or Carrier)

According to an exemplary embodiment of the invention a medicine-delivery lozenge designed for multi-modal absorption in the body of a user, such as a human subject, includes a lozenge base (also referred to as a carrier) sized and formulated for safe oral administration into and manipulation in the mouth of the user. The individual user may either speed up delivery of the active ingredient or slow the release by enhancing or slowing dissolution of ingredients from the delivery system. Rapid release is usually accomplished by enhanced chewing, licking or sucking whereas slower release is accomplished by the opposite actions.

Lozenge bases are flavored dosage delivery systems for medicament(s) that are held in the mouth, wetted with saliva and sucked until dissolution occurs. A wide variety of lozenge bases can be used as the multi-modal drug delivery vehicle for medicament(s) and buffer chemicals. Generally, lozenge bases have a base composed of a mixture of sugar and other carbohydrate bulking agents. Non-fermentable sugars such as sorbitol, mannitol, xylitol, isomalt and hydrogenated starch hydrolysates may also be used. A general discussion of lozenge forms of confectionery may be found in H. A. Lieberman, Pharmaceutical Dosage Forms, Volume 1: Tablets (1989), Marcel Dekker, Inc., New York, N.Y. at Medicated Confections, pages 419-582, which disclosure is incorporated herein by reference.

The bulk sweeteners may constitute, for example, about 20 weight percent to about 80 weight percent of the total weight of the lozenge and may include both sugar and sugarless sweeteners. Such ingredients are well known in the art and are selected to impart improved palatability and to aid in masking the bitter or unpleasant taste of some medicaments. High intensity and artificial sweeteners may also be included such as saccharin and its various salts, cyclamic acid and its various salts, sucralose, aspartame, acesulfame K and other high-intensity sweeteners known in the art.

The lozenge base may further contain gums, thickeners, and hydrophilic polymers such as in a range of about 0.5 to about 20 weight percent of the total weight of the lozenge. The gums and thickeners may act as a mucosal adhesive. Examples include hydroxyproplycellulose, hydroxypropyl methylcellulose, sodium alginate, xanthan gum, calcium polycarbophil, guar gum, carrageenan, gum arabic, locust bean gum, polyvinyl acetate, polyvinyl alcohol and combinations thereof.

The lozenge base may still further contain lubricants and glidants. Representative amounts range from about 0.5 to about 5 weight percent based on the total weight of the lozenge. Magnesium stearate, calcium stearate, zinc stearate, hydrogenated vegetable oils, sterotex, polyoxyethylene, monostearate, talc, polyethylene glycol, sodium benzoate, sodium lauryl sulfate, magnesium lauryl sulfate, calcium silicate, magnesium oxide, magnesium carbonate, and light mineral oil are just a few examples of lubricants and glidants that may be used.

In addition, flavorants and other excipients may be used in the lozenge base. The flavorants constitute, for example, about 0.1 to about 10 weight percent, more preferably about 0.5 to about 4 weight percent of the total weight of the lozenge. The flavoring agents may include natural and synthetic agents and all such combinations thereof. Colorants may include food and pharmaceutical grade coloring agents.

Additional ingredients may also be included in the lozenge, such as antioxidants. In some cases, it may be desirable to include an antioxidant to protect the flavorants and active ingredients from oxidation.

Active Medicinal Ingredient

The lozenge further includes one or more active medicinal ingredients. Preferably the active medicinal ingredient is dispersed substantially uniformly in the lozenge base, although alternatively the active medicinal ingredient may be concentrated, for example, in the core or a coating of the lozenge. The overall concentration of the active medicinal ingredient generally will depend upon many factors, including the desired potency of the lozenge and the intended user, e.g., lower dosages for children than adults. Generally, the active medicinal ingredient may constitute about 0.05 to about 10.0 weight percent of the total weight of the lozenge, although amounts outside this range may be practiced within the scope of the invention.

In exemplary embodiments of the invention the active medicinal ingredient is a non-sedating antihistamine. Suitable antihistamines include, for example, loratadine, desloratadine, fexofenadine, hydroxyzine, cetirizine, and combinations thereof, and other active ingredients capable of relieving histamine symptoms and allergic responses and achieving other like therapeutic effects. The antihistamine has limited water solubility and sufficient lipophilicity for facilitating both transmucosal absorption from the oral cavity and absorption from the gastro-intestinal tract. For example, loratadine has a water solubility from about 0.004 to about 0.006 mg/mL @ pH 6.5. Also, loratadine has a suitable lipophilicity with Log P of 5.2 (octanol/H₂O).

The lozenge further may contain one or more secondary active medicaments for relieving other related symptomatology from allergic responses such as bronchodilators and decongestants. Examples of medicaments which may be further included in the lozenge for promoting bronchodilation and/or decongestion are pseudoephedrine, dextromethorphan, guiafensin, aspirin, acetaminophen, doxylamine, diphenhydramine, and combinations thereof. The secondary active medicament(s) may be present in an amount of, for example, about 3 to about 12 weight percent based on the total weight of the lozenge. The secondary medicament preferably is principally absorbed from the gastro-intestinal tract. The lozenge maybe formulated for immediate release or for controlled release of the secondary active medicament(s).

Dissolution of the medicament(s) may be enhanced by increasing its surface area by micronization (<15 microns) and by encapsulation in a water soluble matrix using spin head to form flakes, as discussed in U.S. Pat. No. 5,458,823. Encapsulation also may be used for achieving multi-phasic release of the active medicinal ingredient. Emulsifiers can also be used to enhance the dissolution and increase the solubility of the medicament(s). For example, common food grade emulsifiers such as monoglycerides, monoglyceride derivatives, sorbitan derivatives (polysorbate 60, 65, and 80), polyglycerol esters, anionic emulsifiers (sodium stearoyl lactylate, sodium stearyl fumarate, sodium dodecyl sulfate), and lecithin may be selected. If present, an effective amount of emulsifier may range from, for example, about 0.05 to about 10 weight percent based on the total weight of the lozenge.

Buffer

The ratio of non-ionized active ingredient to ionized active ingredient in the mouth can be shifted to favor absorption in the mouth. This can be accomplished by release of buffer chemicals into the mouth from the delivery vehicle that alters the pH of saliva and subsequently, the amount of active ingredient in the non-ionized form that can be absorbed transmucosally.

The lozenge includes one or more buffers in an amount selected to produce a desired pH, such as in the range of about 4 to about 10, in the user's mouth. Preferably the buffer is dispersed substantially uniformly in the lozenge base, although alternatively the buffer may be concentrated, for example, in the core or a coating of the lozenge. Generally, the base may constitute about 4 to about 50 weight percent of the total weight of the lozenge, although amounts outside this range may be practiced within the scope of the invention.

In certain exemplary embodiments the body's absorption of the active medicinal ingredient(s) contained in the carrier occurs in multiple modes. The first mode or phase comprises a rapid absorption of the active medicinal ingredient into the bloodstream by a transmucosal route (sublingual, buccal, pharyngeal). Transmucosal absorption of the active is facilitated by a pH adjustment produced by the buffer(s) in the lozenge. The transmucosally absorbed first portion (also referred to as the first pharmacologically effective dose) of active medicinal ingredient(s) rapidly reaches the bloodstream to provide fast and highly efficacious relief to the targeted health problem(s), such as histamine effects and symptoms. The second mode or phase comprises the gastrointestinal absorption of a second portion (also referred to herein as a second pharmacologically effective dose) of the active medicinal ingredient(s) swallowed by the user. Because the second dose travels the gastrointestinal tract, it is absorbed into the bloodstream at a slower rate than the first dose.

The release of buffers from the lozenge controls the pH in the user's mouth, which in turn affects the amount of active medicinal ingredient which is transmusocally absorbed. Many active medicinal ingredients contemplated for use in the embodied lozenge are sensitive to pH conditions. It is well known that drug absorption across biological membranes (sublingual, buccal, pharyngeal) occurs primarily by passive diffusion of non-ionized drug. Drugs that are either acidic or basic can exist in both ionized and non-ionized forms. The degree of ionization is dependent upon the chemical properties of the drug and the pH of the environment. Medicaments that contain basic nitrogen moieties in their structure, referred to herein as weak bases, may demonstrate a pKa in the range of 3 to 11. The degree of ionization of a specific medicament at a specific pH condition can be predicted by use of the Henderson-Hasslebach equation. For weak bases, the Henderson-Hasselbach can be expressed as follows:

pH=pKa+log [(non-ionized base)/(ionized base)]

Under acidic pH conditions in the mouth (pH 6.0 to pH 7.0), those compounds with pKa<7 will exist as both ionized and non-ionized drug. Acidic buffer chemicals such as citric acid combined with sodium citrate rapidly lower the pH of saliva in the mouth and provide favorable conditions for dissolution of weak bases with limited solubility while at the same time allowing a portion of the drag to exist in the non-ionized form necessary for transmucosal absorption of active ingredients. For example, use of sodium citrate and citric acid buffers, which are particularly useful with actives such as loratadine, alter the mouth pH conditions to approximately 4-7 and provide a suitable environment for efficient absorption of most active ingredients containing basic nitrogen groups. Examples of other acidic buffer chemicals for lowering mouth pH conditions are citric, malic, furmaric, tartaric, adipic, and succinic acids, acid anhydrides and acid salts and mixtures thereof. Salts may include, for example, nontoxic levels of potassium, sodium, calcium, magnesium or aluminum.

On the other hand, under acidic pH conditions in the mouth (pH 6.0 to pH 7.0), those compounds with pKa≧7 are primarily ionized and are not efficiently absorbed into the bloodstream by the transmucosal route. For these compounds, basic buffer chemicals such as alkali carbonates rapidly elevate the pH of saliva in the mouth, and provide favorable pH conditions for basic medicaments with pKa≧7 to exist predominantly in the non-ionized form allowing for efficient absorption of active ingredients. For example, use of potassium carbonate buffer in the delivery vehicle alters the mouth pH conditions to approximately 7-10 and provides a suitable environment for efficient absorption of most active ingredients containing basic nitrogen groups. Examples of such basic buffer chemicals are potassium carbonate, potassium bicarbonate, sodium carbonate, sodium bicarbonate, potassium citrate, sodium citrate, calcium carbonate, calcium phosphate, magnesium hydroxide, magnesium carbonate, magnesium trisilicate, aluminum carbonate and aluminum hydroxide, and combinations thereof. For example, citric acid and sodium citrate are suitable buffers for use with loratadine.

In preferred embodiments of the invention the buffer is released simultaneously with the active medicinal ingredient(s) for facilitating transmucosal absorption of active medicinal ingredient(s) from the oral cavity. As a result, a substantial portion of active ingredient is in its non-ionized form and is absorbed quickly. Ionized active medicinal ingredient and un-dissolved medicinal ingredient is swallowed and absorbed into the bloodstream slower via the gastrointestinal tract. As a result, the gastrointestinally absorbed active medicinal ingredient continues to provide the user with long-term relief after the transmucosally absorbed dose has been exhausted. The secondary medicament, such as a bronchodilator or decongestant, if present, may be absorbed transmucosally for immediate relief and/or via the gastro-intestinal tract for prolonged relief.

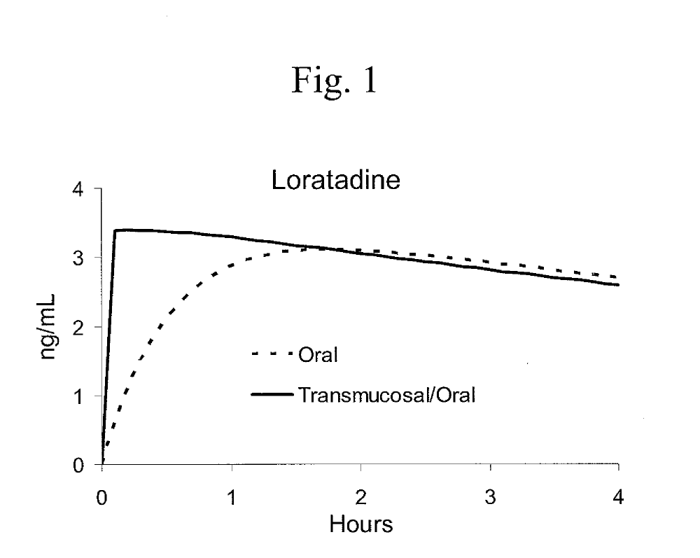

The speed of absorption of the active medicinal ingredient(s) is particularly important because a slow release rate would result in an insufficient amount being absorbed into the bloodstream for early relief of symptoms, conditions or allergic reactions. For example, as shown in FIG. 1, a medicament is absorbed into the bloodstream more rapidly when one portion of the dose is delivered transmucosally and the other portion is delivered gastrointestinally compared to when the entire portion of the medicament is delivered gastrointestinally.

The rates of transmucosal and gastrointestinal absorption will depend upon the amount of time required for the lozenge to dissolve and release the active medicinal ingredient. Generally, lozenges will dissolve, for example, over a period of 5 to 15 minutes from administration into the oral cavity. It is particularly useful for the lozenge to release about 25 to about 60 weight percent of the active medicinal ingredient within the first five minutes of administration. A first pharmacologically effective dose of the non-ionized form of the medicinal active ingredient released from the lozenge base is transmucosally absorbed and the remaining portion (or second pharmacologically effective dose) of the ionized form of the released medicinal active ingredient is swallowed and absorbed in the gastrointestinal tract. It should be understood that the first and second doses may be released from the lozenge simultaneously.

Active medicinal ingredient which has been transmucosally absorbed enters the bloodstream quickly. This first dose may provide rapid relief of histamine effects within as short of a period as 5 to 30 minutes. On the other hand, remaining portion (or second pharmacologically effective dose) of the ionized form of the released medicinal active ingredient is swallowed and absorbed in the gastrointestinal tract over the next 1-4 hours. This second dose may provide sustained relief over a prolonged period, e.g., of 12-24 hours. By way of example, the first pharmacologically effective dose transmucosally absorbed may constitute about 10 to about 80 weight percent, more preferably about 20 to about 80 weight percent, and still more preferably about 40 to about 75 weight percent of the active medicinal ingredient. The second pharmacologically effective dose gastro-intestinally absorbed may constitute, for example, about 20 to about 90 weight percent, more preferably about 20 to about 80 weight percent, and still more preferably about 25 to about 60 weight percent of the active medicinal ingredient.

The multi-modal absorption, medicine-delivery lozenge described herein is particularly useful for oral administration to attain relief from histamine effects and symptoms in individuals, especially humans, in need of relief of histamine effects and symptoms The multi-modal absorption, medicine-delivery lozenge described herein is also particularly useful for oral administration to attain relief from allergic responses in individuals, especially humans, in need of relief from allergic responses. The dosage amount will vary depending upon the weight, age, and needs of the individual. Generally, a single dosage is sufficient for a 24 hour period, although the dosage may be increased as dictated by the individual's symptoms and response to the lozenge. The lozenge may be packaged with administration instructions for attaining the desired relief

Standard processing equipment and blending and pressing techniques may be used in the manufacture of the lozenge. The bulk powder (e.g., mannitol, sorbitol) may be granulated to improve its flowability, such as by practicing wet mixing, granulation, and drying steps. Alternatively, dry compaction also may be practiced. Tablet pressing equipment may be employed for carrying out the compression step.

Various changes and modifications to the embodiments of the invention described above will be apparent to persons skilled in the art having reference to this disclosure.

While this invention has been described as having exemplary embodiments, it is understood that the invention is not limited to the illustrated and described features. To the contrary, the invention is capable of further modifications, uses, and/or adaptations following the general principles of the invention and therefore includes such departures from the present disclosure as come within the known or customary practice in the art to which the invention pertains, and as may be applied to the central features set forth above.

What is claimed is:

1. A multi-modal absorption, medicine-delivery lozenge, comprising:
a lozenge base for oral administration into a user's mouth;
a medicinal active ingredient comprising an antihistamine convertible between ionized and non-ionized forms; and
a buffer present in an effective amount for establishing a pH in the user's mouth which is characterized by a simultaneous presence of a first pharmacologically effective dose of the non-ionized form of the medicinal active ingredient for transmucosal absorption, and a second pharmacologically effective dose of the ionized form of the medicinal active ingredient for gastro-intestinal absorption.

2. The multi-modal absorption, medicine-delivery lozenge of claim 1, wherein the lozenge base possess a composition which dissolves in the user's mouth over a period of 5 to 15 minutes.

3. The multi-modal absorption, medicine-delivery lozenge of claim 1, wherein the medicinal active ingredient comprises a member selected from the group consisting of desloratadine, fexofenadine, bydroxyzine, cetirizine, and combinations thereof.

4. The multi-modal absorption, medicine-delivery lozenge of claim 1, wherein the medicinal active ingredient comprises loratadine.

5. The multi-modal absorption, medicine-delivery lozenge of claim 1, wherein medicinal active ingredient and the buffer are substantially uniformly dispersed in the lozenge base.

6. The multi-modal absorption, medicine-delivery lozenge of claim 1, wherein the buffer establishes a pH of approximate 4 to 7 in the user's mouth.

7. The multi-modal absorption, medicine-delivery lozenge of claim 1, wherein the first pharmacologically effective dose is sufficient to provide rapid relief of histamine effects within 5 to 30 minutes after transmucosal absorption.

8. The multi-modal absorption, medicine-delivery lozenge of claim 1, wherein the second pharmacologically effective dose is sufficient to provide prolonged relief of histamine effects over a period of 12 to 24 hours.

9. The multi-modal absorption, medicine-delivery lozenge of claim 1, further comprising an additional medicament selected from the group consisting of a bronchodilator and a decongestant.

10. The multi-modal absorption, medicine-delivery lozenge of claim 1, wherein a portion of the medicinal active ingredient is microencapsulated for attaining multi-phasic release of the medicinal active ingredient.

11. The multi-modal absorption, medicine-delivery lozenge of claim 1, wherein the first pharmacologically effective dose transmucosally absorbed constitutes about 40 to about 75 weight percent of the active medicinal ingredient, and the second pharmacologically effective dose gastro-intestinally absorbed constitutes about 25 to about 60 weight percent of the active medicinal ingredient.

12. The multi-modal absorption, medicine-delivery lozenge of claim 1, wherein the first pharmacologically effective dose transmucosally absorbed constitutes about 10 to about 80 weight percent of the active medicinal ingredient, and the second pharmacologically effective dose gastro-intestinally absorbed constitutes about 20 to about 90 weight percent of the active medicinal ingredient.

13. A method of attaining relief from histamine effects and symptoms, comprising:
orally administering a multi-modal absorption, medicine-delivery lozenge to an individual in need of relief of histamine effects and symptoms, the lozenge comprising a lozenge base for oral administration into the individual's mouth, a medicinal active ingredient comprising an antihistamine convertible between ionized and non-ionized forms, and a buffer;
establishing a pH in the individual's mouth which is characterized by a simultaneous presence of a first pharmacologically effective dose of the non-ionized form of the medicinal active ingredient for transmucosal absorption, and a second pharmacologically effective dose of the ionized form of the medicinal active ingredient for gastrointestinal absorption; and
permitting the medicinal active ingredient to cause relief from histamine effects and symptoms.

14. The method of claim 13, wherein the medicinal active ingredient comprises a member selected from the group consisting of desloratadine, fexofenadine, hydroxyzine, cetirizine, and combinations thereof.

15. The method of claim 13, wherein the medicinal active ingredient comprises loratadine.

16. The method of claim 13, wherein medicinal active ingredient and the buffer are substantially uniformly dispersed in the lozenge base.

17. The method of claim 13, wherein a portion of the medicinal active ingredient is microencapsulated for attaining multi-phasic release of the medicinal active ingredient.

18. The method of claim 13, wherein the first pharmacologically effective dose transmucosally absorbed constitutes about 40 to about 75 weight percent of the active medicinal ingredient, and the second pharmacologically effective dose gastro-intestinally absorbed constitutes about 25 to about 60 weight percent of the active medicinal ingredient.

19. The method of claim 13, wherein the first pharmacologically effective dose transmucosally absorbed constitutes about 10 to about 80 weight percent of the active medicinal ingredient, and the second pharmacologically effective dose gastro-intestinally absorbed constitutes about 20 to about 90 weight percent of the active medicinal ingredient.

20. A method of attaining relief from allergic responses, comprising:
orally administering a multi-modal absorption, medicine-delivery lozenge to an individual in need of relief of allergic responses, the lozenge comprising a lozenge base for oral administration into the individual's mouth, a medicinal active ingredient comprising an antihistamine convertible between ionized and non-ionized forms, and a buffer;
establishing a pH in the individual's mouth which is characterized by a simultaneous presence of a first pharmacologically effective dose of the non-ionized form of the medicinal active ingredient for transmucosal absorption, and a second pharmacologically effective dose of the ionized form of the medicinal active ingredient for gastro-intestinal absorption;
permitting the medicinal active ingredient to cause relief from allergic responses.
